(19) United States
(12) Patent Application Publication
(10) Pub. No.: US 2020/0087621 A1
(43) Pub. Date: Mar. 19, 2020

(54) Glycosaminoglycan Mimetic Scaffolds

(71) Applicants: New Jersey Institute of Technology, Newark, NJ (US); University of Miami, Miami, FL (US)

(72) Inventors: Treena Lynne Arinzeh, West Orange, NJ (US); Roseline Menezes, Newark, NJ (US); George Collins, Maplewood, NJ (US); Sharareh Hashemi, Raritan, NJ (US); Martin Oudega, Miami, FL (US)

(73) Assignees: New Jersey Institute of Technology, Newark, NJ (US); University of Miami, Miami, FL (US)

(21) Appl. No.: 16/573,206

(22) Filed: Sep. 17, 2019

Related U.S. Application Data
(60) Provisional application No. 62/732,075, filed on Sep. 17, 2018.

(51) Int. Cl. C12N 5/079

(52) U.S. Cl. CPC C12N 5/0622; C07K 2318/20; C12N 2533/70; C12N 2533/90; C12N 2535/00

(57) ABSTRACT

Sodium cellulose sulfate (NaCS) is employed as a novel GAG mimetic. Schwann cells (SCs) could be used in combination with a scaffold because the SCs can secrete neurotrophic factors stimulating neuron survival and extension of axons. Furthermore, the conduit may be used alone or combination with Schwann cells for spinal cord repair. In addition, the conduit also can be used for peripheral nerve repair. Also described herein are compositions and methods useful for promoting the growth and/or differentiation and/or repair of a cell and/or tissue in the peripheral nervous system, central nervous system, and specifically the spinal cord. In certain aspects, the present disclosure includes a scaffold supporting and promoting growth, differentiation, and/or regeneration and repair. The scaffold in one embodiment closely mimics the natural extracellular matrix (ECM) of the spinal cord.

### CROSS-REFERENCE TO RELATED APPLICATIONS

The present application claims the benefit of the filing date of U.S. Provisional Patent Application No. 62/732,075 filed on Sep. 17, 2018 the disclosure of which is hereby incorporated herein by reference.

### FIELD

The present disclosure generally relates to biotechnology and regenerative medicine. In particular, the present disclosure is directed to a glycosaminoglycan (“GAG”) mimetic scaffold containing aligned fibers for peripheral nerve and spinal cord repair.

### BACKGROUND

According to the World Health Organization, more than a half-million people each year suffer from some type of spinal cord injury (SCI). In the USA alone, there are 17,000 new SCI cases each year. People with a spinal cord injury are two to five times more likely to die prematurely than people without a spinal cord injury, with worse survival rates in low-income and middle-income countries. Spinal cord injury is associated with lower rates of school enrollment and economic participation, and it carries substantial individual and societal costs.

Brain injuries, cranial nerve damage, peripheral nerve damage, traumatic nervous tissue lesions and neurodegeneration drastically reduce life quality and lead to severe and often fatal impairments, largely because the central nervous system (CNS) of adult mammals retains a low capacity for regeneration into adulthood. The CNS regulates the function of various organs. Injury to the CNS causes impairment of neurological functions in corresponding sites and further leads to long-term patient disability.

Generally, nervous tissue regeneration involves the replacement of lost neurons (de novo neurogenesis) and/or the repair of damaged axons (axonal regeneration). Functional deficits persist after SCI, traumatic brain injury, stroke, and related conditions that involve axonal disconnection. This situation differs from that in the mammalian peripheral nervous system (PNS), where long-distance axon regeneration and substantial functional recovery can occur in the adult.

Two known major classes of CNS regeneration inhibitors are the myelin-associated inhibitors (MAIs) and the chondroitin sulfate proteoglycans (CSPGs). MAIs are proteins expressed by oligodendrocytes as components of CNS myelin. MAIs impair neurite outgrowth in vitro and are thought to limit axon growth in vivo after CNS damage. MAIs include, for example, Nogo-A, myelin-associated glycoprotein (MAG), oligodendrocyte myelin glycoprotein (OMgp), ephrin-B3, and Semaphorin 4D (Sema4D).

Typically, a so-called “glial scar” forms after CNS injury. Glial scar formation (gliosis) is a reactive cellular process involving astrogliosis that occurs after injury to the central nervous system. As with scarring in other organs and tissues, the glial scar is the body's mechanism to protect and begin the healing process in the nervous system. In addition, this scar is a physical barrier to regeneration and also contains inhibitory molecules that impede axon growth. CSPGs are the main inhibitory molecules found in the glial scar and are upregulated by reactive astrocytes after CNS damage. CSPGs can be both membrane bound and secreted into the extracellular space. CSPG inhibitors include, for example, neurocan, versican, brevican, phosphacan, aggrecan, and NG2. Other CNS axon regeneration inhibitors (ARIs) that are not present in myelin or the glial scar include repulsive guidance molecule (RGM) and semaphorin 3A.

CNS regeneration is difficult because of its poor response to treatment and, to date, no effective therapies have been found to rectify CNS injuries. Biomaterial “scaffolds” have been applied with promising results in regeneration medicine. They also show great potential in CNS regeneration for tissue repair and functional recovery; however, current scaffolds still have significant drawbacks as the compositions are stopped by the above inhibitors ARI and other molecules that impede axon growth.

One general approach to the use of tissue engineering in the repair and/or regeneration of tissue is to combine cells and/or biological factors with a biomaterial that acts as a scaffold for tissue development. The cells should be capable of propagating on the scaffold and acquiring the requisite organization and function to produce a properly functioning tissue. Recent attempts to address this health need have been met with limited success.

Spinal cord injury is a devastating heterogeneous neurological condition with no effective treatment at the present time. Despite recent investigation of several promising therapeutic avenues, no therapies have been found for the safe and cost-effective treatment that leads to long-term attenuation to date. Accordingly, a need exists to develop compositions and methods that are capable of promoting spinal cord growth and repair. There still exists a critical need for a composition and method for treating peripheral nerve damage and promotes peripheral nerve repair in general. There is also a need for a treatment that promotes growth, differentiation, and/or regeneration and repair and mimics the natural extracellular matrix (ECM) of the spinal cord.

### SUMMARY

The present disclosure solves the above recited drawbacks and problems of current state of the art and provide many more benefits as described herein. The composition and method of the present invention may be used in a variety of applications that involve peripheral nerve repair in general, and more specifically promote spinal cord growth and repair.

Described herein are compositions and methods useful for promoting the growth and/or differentiation and/or repair of a cell and/or tissue in the peripheral nervous system. In certain aspects, the present disclosure includes a scaffold supporting and promoting growth, differentiation, and/or regeneration and repair. The scaffold in one embodiment closely mimics the natural extracellular matrix (ECM) of the spinal cord.

The ECM provides the environment to execute cellular processes responsible for cellular replication, differentiation, maturation, and survival. These processes require profuse cell communication and the biological interplay between cell receptors and protein factors. Glycosaminoglycan (GAG) that is present in native tissue provides signaling and structural cues to cells. GAGs are sulfated polysaccharides that are constituent components of the ECM and have been implicated in the stabilizing biological activity of protein factors, as well as facilitating the interaction of protein factors with cell receptors. It was presently found that glycosaminoglycans (GAGs), such as chondroitin sulfate (CS), can either inhibit or promote axonal growth depending upon the degree and pattern of sulfation.

In accordance with embodiments of the present disclosure, exemplary glycosaminoglycan (GAG) mimetics are used as scaffolds. In one embodiment, sodium cellulose sulfate (NaCS) is employed as a novel GAG mimetic. NaCS can be tailored to have varying degree and pattern of sulfation similar to native GAGs, CS-C and CS-D. Chondroitin sulfate-C (CS-C) is chondroitin-6-sulfate and chondroitin sulfate-D (CS-C) is chondroitin-2,6-sulfate. The position of the sulfate is indicated by the number. Schwann cells (SCs) could be used in combination with this scaffold since they secrete neurotrophic factors stimulating neuron survival and extension of axons.

In one embodiment, a method to promote spinal cord growth and repair is provided. NaCS with varying degree of sulfation is dissolved in deionized (DI) water and mixed with a solution of either 100% bovine gelatin or 80:20 ratio of gelatin: poly-caprolactone (PCL) in acetic acid (AA) and 2,2,2-trifluoroethanol (TFE). NaCS is dissolved in 15% DI water using a water bath at 60 degree Celsius, then gelatin and 25% acetic acid are added to the solution and stirred for 2 hours. When adding a synthetic polymer, PCL is dissolved in 60% TFE and the two solutions are mixed. Otherwise, 60% TFE is added to the gelatin solution and stirred. Scaffolds containing aligned fibers are prepared by the electrospinning technique.

Another objective is to improve the current standard of care for injury in the CNS, peripheral nervous system and spinal cord areas.

Another advantage of the present invention is that the newly developed GAG mimetic, sodium cellulose sulfate (NaCS), can be tailored to have varying degrees and patterns of sulfation similar to native GAGs, CS-C and CS-D. Schwann cells (SC) are of interest to be used in combination or not in combination with this scaffold since they secrete neurotrophic factors stimulating neuron survival and extension of axons. The conduit used in the present invention may be used alone or combination with Schwann cells for spinal cord repair. In addition, the conduit also can be used for peripheral nerve repair.

Still another objective is to fabricate, characterize, and evaluate NaCS containing scaffolds containing aligned fibers for supporting SC growth and neurite extension.

Any combination and/or permutation of the embodiments are envisioned. Other objects and features will become apparent from the following detailed description considered in conjunction with the accompanying drawings and claims. It is to be understood, however, that the drawings are designed as an illustration only and not as a definition of the limits of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. To assist those of skill in the art in making and using the disclosed scaffold and associated systems and methods, reference is made to the accompanying figures, wherein:

FIG. 1A illustrates SEM image of a gelatin GAG mimetic scaffold before crosslinking the scaffold;

FIG. 1B illustrates SEM image of a gelatin GAG mimetic scaffold after crosslinking the scaffold;

FIG. 2 is a chart showing an amount of nerve growth factor (NGF) on Gelatin/fully sulfated sodium cellulose (fNaCS) scaffold, with sulfate groups on the 2, 3, and 6th position of glucose unit scaffold, after 24 hours incubation in comparison to only Gelatin, and Gelatin/partially sulfated sodium cellulose (pNaCS) scaffold, with sulfate groups on 6th position of glucose unit scaffold;

FIG. 3A illustrates a confocal fluorescent image of dorsal root ganglion (DRG) with neurofilament stain (green) on Gelatin at a magnification of 4× using a scale bar equal to 250 μm;

FIG. 3B illustrates a confocal fluorescent image of DRG with neurofilament stain (green) on Gelatin/pNaCS at a magnification of 4× using a scale bar equal to 250 μm;

FIG. 3C illustrates a confocal fluorescent images of DRG with neurofilament stain (green) on Gelatin/fNaCS at a magnification of 4× using a scale bar equal to 250 μm;

FIG. 4 is a chart showing Schwann cells (SCs) growth up to 10 days;

FIG. 5A illustrates a confocal fluorescent image of SCs at day 10 on Gelatin at a magnification of 20× and a scale bar equal to 100 μm;

FIG. 5B illustrates a confocal fluorescent image of SCs at day 10 on Gelatin/pNaCS at a magnification of 20× and a scale bar equal to 100 μm;

FIG. 5C illustrates a confocal fluorescent image of SCs at day 10 on Gelatin/fNaCS at a magnification of 20× and a scale bar equal to 100 μm;

FIG. 6 shows a schematic of the fabrication of the sodium cellulose sulfate-Gelatin also known as NaCS-Gelatin or Ce1S-Gelatin Conduits for in vivo studies; and

FIG. 7 illustrate confocal fluorescent images of NaCS-Gelatin based conduits (without Schwann cells (SCs) where the sample size is n of 6 after 1 week of implantation in the rat transected spinal cord.

### DETAILED DESCRIPTION

The present disclosure is directed to novel compositions and methods that overcome the drawbacks of current treatment methodologies and attempts for peripheral nerve and spinal cord repair. The current disclosure facilitates development and exploration of new therapeutic avenues to treat peripheral nerve, CNS, and spinal cord injuries. The current work describes herein, among other things, how a combination of SCs and GAG-mimetic scaffold can be used as a novel approach to effectively promote and direct axonal growth. Significant neurite growth on Gelatin/fully sulfated sodium cellulose scaffold (Gel/fNaCS) suggests that neurite outgrowth can be promoted by the GAG-mimetic and may depend on the degree and pattern of sulfation.

Sodium Cellulose Sulfate (NaCS) is a semi-synthetic polysaccharide with a similar structure to GAGs. The use in this Description, attached Figures, and claims of the abbreviation “NaCS” is used interchangeably with “Ce1S” to mean Sodium Cellulose Sulfate. NaCS is derived from cellulose, and can be synthesized to include up to three sulfate groups per glucose, which may be beneficial for growth factor binding. The present inventors evaluated sodium cellulose sulfate (NaCS) containing scaffolds containing aligned fibers for supporting Schwann cells growth and neurite extension. Furthermore for the purposes of this Description, Figures, and claims partially sulfated sodium cellulose sulfate (pNaCS), and fully sulfated sodium cellulose sulfate (fNaCS) are used interchangeably with pNaCe1S and fNaCe1S, as well as pCe1S and fCe1S respectively.

The materials and the methods of the present disclosure used in one embodiment will be described below. While the embodiment discusses the use of specific compounds and materials, it is understood that the present disclosure could employ other suitable materials. Similar quantities or measurements may be substituted without altering the method embodied below.

The following examples illustrate the features of the invention. In no way is the following examples meant to limit the scope of the invention to a particular embodiment. Each example is merely given as one aspect of the invention and to illustrate its desired properties.

In one embodiment, aligned fibers were prepared by electrospinning a solution of 24% (w/v) of gelatin (obtained from Bovine skin, Sigma) with 0.25% (w/w) GAG mimetics in 60% TFE (2,2,2-Trifluoroethanol), 25% AA (Glacial acetic acid), and 15% DI water (v/v %). Two types of GAG mimetics, partially sulfated sodium cellulose (pNaCS), with sulfate groups on 6th position, and fully sulfated sodium cellulose (fNaCS), with sulfate groups on the 2, 3, and 6th position of glucose unit, were used. Depending on the embodiment, other types of GAG mimetics could be used and the location of the sulfate groups for pNaCS and fNaCS could vary.

For electrospinning aligned fibers, a grounded rotating mandrel was used as a collector. The scaffolds were then crosslinked using N-(3-dimethyl aminopropyl)-N′-ethyl carbodiimide hydrochloride (EDC) with N-hydroxysulfosuccinimide (NHS) (EDC/NHS). It will be understood that other cross-linkers could be used.

Fiber morphology and alignment of the scaffolds were evaluated using SEM. GFP labeled SCs were seeded onto scaffolds at 7×10⁴ cells/cm² and cultured for up to 10 days. At days 1, 4, 7, and 10, cell number (n=4) was determined using PicoGreen assay (Invitrogen). Cell attachment (n=2) was observed by confocal microscopy. Dorsal root ganglion (DRG) isolated from the spinal cord of 16-day-old Sprague-Dawley rat embryos (E-16) was seeded on scaffolds. After 4 days in culture, neurite extension and neurofilament immunostaining was viewed by confocal microscopy. Neurotrophin binding was determined by incubating scaffolds in 20 ng/ml of nerve growth factor (NGF) for 24 hours at 37° C.

In another embodiment, the following fabrication methods and in vivo studies were conducted.

Fabrication of aligned fibrous scaffolds: In this example, scaffolds are fabricated using electrospinning technique. Polymer solutions are prepared by dissolving polymers in appropriate solvents. Aligned fibrous scaffolds are collected on a rotating grounded mandrel. Depending on the embodiment, the mandrel speed may be at a speed of about 3200 rpm. Scaffolds are aerated under a hood for a week for solvent evaporation. The average scaffolds thickness is about 0.2 mm. Other thicknesses may be used depending on the implementation.

Partially sulfated cellulose sulfate (pNaCS) with 0.5 sulfates per glucose unit are prepared as previously known, and fully sulfated cellulose sulfate (fNaCS) with 3 sulfates per glucose unit from Dextran Products Limited, Ontario, Canada are used. The solutions of either 25% w/v gelatin (bovine skin type B, Sigma Aldrich, USA) or 22% w/v gelatin/polycaprolactone (Gel/PCL) (Mₙ=80,000, Sigma Aldrich, USA) at the ratio of 80:20 with 0.25% of fNaCS or pNaCS will be prepared in trifluoroethanol (TFE): acetic acid (AA): deionized water at the ratio of 60:25:15. The sodium cellulose sulfate (NaCS) concentration of 0.25% was chosen based on the sulfated GAGs present in decellularized neural tissue in rats as previously known.

NaCS was dissolved in water for lhr in a water bath at 55° C., and Gel added along with AA and stirred for another 2 hours while it is still in the water bath. The solution was cooled down at room temperature for few minutes and TFE added and stirred overnight at room temperature. In the case of Gel/PCL solutions, PCL was dissolved in TFE at room temperature separately for 3 hours and mixed with Gel solution. Gelatin alone or Gel/PCL scaffolds alone are also prepared this way.

The above solvents were chosen so that scaffolds could be prepared containing PCL. Prepared was Gelatin alone (Gel) and Gel/NaCS scaffolds using other solvents as known in the art. A solution of 20-30% w/w bovine gelatin can be mixed with 0.25% (w/w) NaCS in 50:50 ratio of deionized water to ethanol (200 proof, Sigma) at 60° C. in order to prevent the gelatin from gelling at room temperature. Acetic acid may also be used as a substitute for ethanol in this formulation.

Conduit fabrication: The conduit or rolled scaffold was made in this embodiment as follows. Gel/NaCS and Gel/PCL/NaCS scaffolds were cut at the desired width depending on the desired final diameter of conduit. For example, a 1.5 mm and 1.8 mm width for Gel and Gel/PCL, respectively, for a rat spinal cord was made and corresponding desired length.

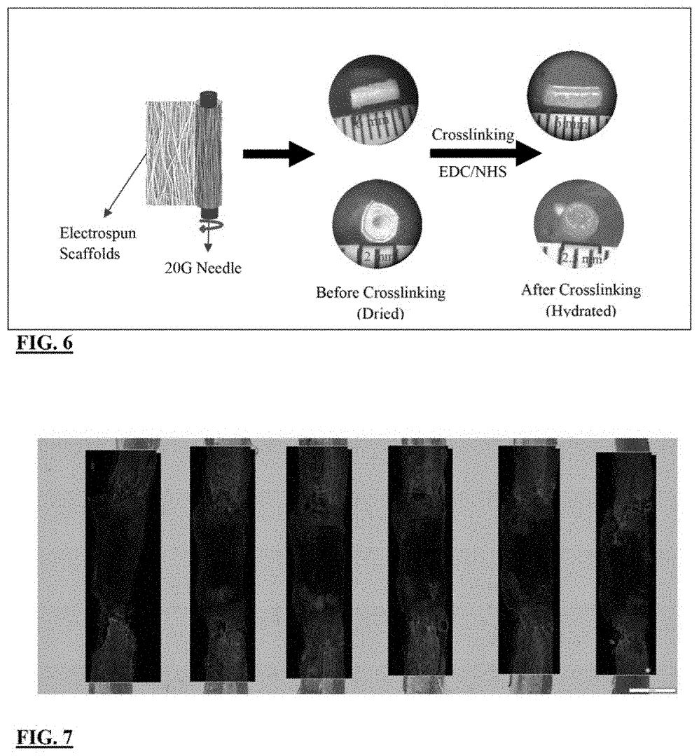

In this example, a 20 gauge (G) stainless steel needle (outside diameter-O.D.: 0.9 mm) was used to roll the scaffolds gently longitudinally around the needle to form a spiral shape conduit. This rolling is also observed in FIG. 6. The last layer of the rolled scaffold was sealed longitudinally using deionized (DI)-water to avoid unfolding during implantation. It was noted that adding too much water will dissolve scaffolds. Since the Gel is not crosslinked yet, it will dissolve easily in water. Care was taken as not to dissolve the rolled scaffold or conduit. This part of the procedure should be done very gently otherwise it can easily form holes along the length or stick all layers together. The needle is removed gently to avoid disturbing the fiber alignment.

The newly formed “conduit” or rolled scaffold was crosslinked using N-(3-dimethyl aminopropyl)-N′-ethyl carbodiimide hydrochloride (EDC) with N-hydroxysulfosuccinimide (NHS) crosslinking method for 96 hrs to improve their hydrolytic stability in physiological condition. The conduit was sterilized using series of ethanol washes (50%, 70%, and 100% ethanol) and stored.

In Vivo Study and Placement in Spinal Cord: For In Vivo study, the conduits are hydrated in phosphate buffered saline (PBS) for 2 hours, pretreated with media overnight, and loaded with Schwann cells prior implantation. For In Vivo placement in the Spinal Cord, rats were anesthetized and a laminectomy performed at thoracic levels 8 (T8) to expose the underlying T9 spinal cord as known in the art. The dura was gently opened and a 3 mm-long segment of the spinal cord removed using surgical micro-scissors. Retraction of the caudal spinal cord stump created a 5 mm gap. After bleeding was controlled, a 5 mm-long fCe1S-Gelatin conduit was placed in the gap making sure that the rostral and caudal spinal cord stumps are minimally manipulated and gently opposed to the ends of the implant.

The conduit and its interfaces with the rostral and caudal spinal cord was covered with artificial dura, such as a known silicone sheathing, before closing the muscles individually with sutures and closing the skin with metal Michel wounds clips.

The rat model is a completely transected spinal cord model to allow evaluation of the regeneration of nerves. The conduit/graft design may also be used for a contused spinal cord that is a prevalent injury seen clinically.

Results

The results of the experiment for the present embodiment will now be discussed.

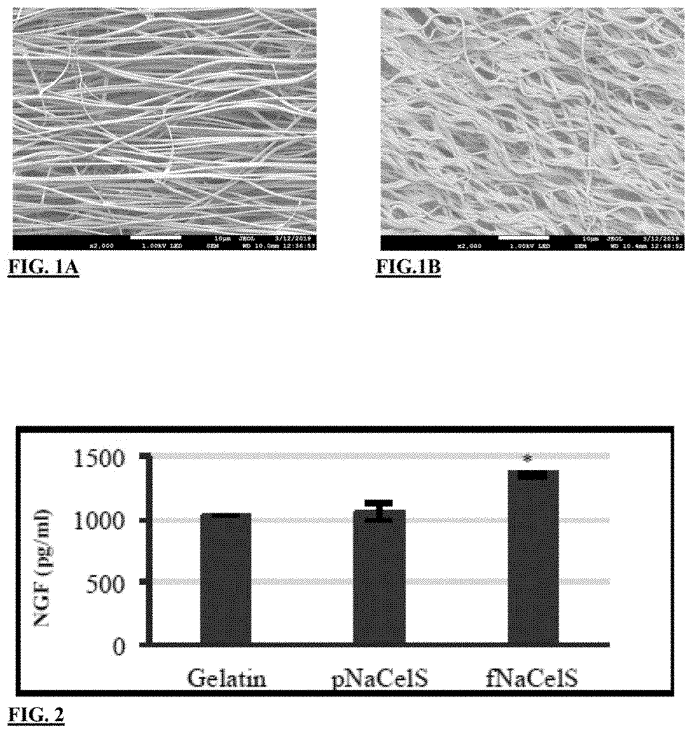

As shown in FIGS. 1A-1B, the scaffolds had a uniform morphology with an average fiber diameter of 1 μm with approximately 80% alignment maintaining after crosslinking. Shown are SEM images of Gelatin scaffolds (Mag 2000×) before and after crosslinking.

Referring to FIG. 2, a significantly higher amount of NGF was determined on Gel/fNaCS scaffold in comparison to both Gel and Gel/pNaCS. The graph illustrates the amount of nerve growth factor (NGF) after 24 hrs incubation. In this comparison, the sample size n=3; and with a statistical significance p<0.05 in comparison to Gel and pNaCe1S. Again, pNaCe1S is used as the same term to mean pNaCS for partially sulfated sodium cellulose. In addition NaCe1S is used as the same term for NaCS for sulfated sodium cellulose. Data is shown as mean±Standard deviation.

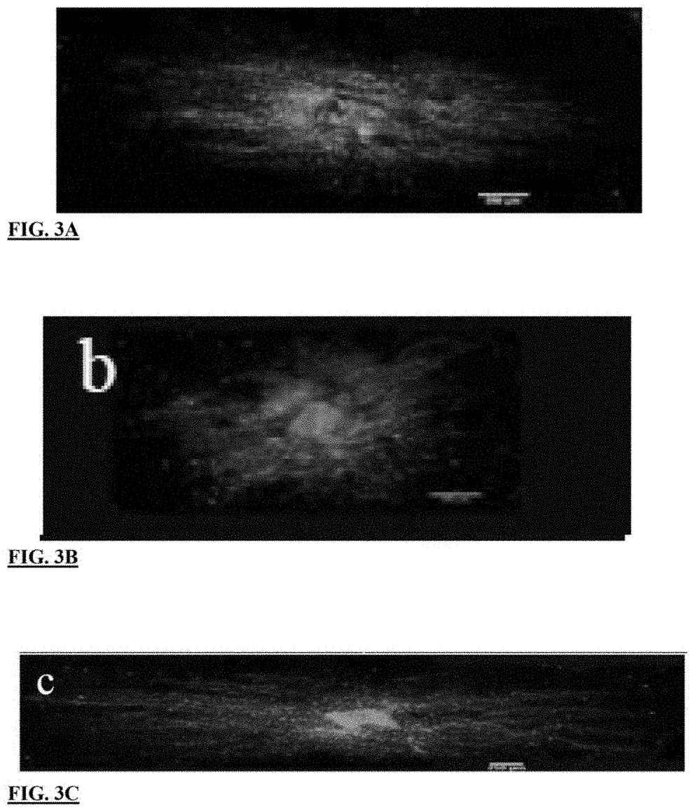

Turning now to FIGS. 3A, 3B, and 3C, a significantly higher neurite length was determined for dorsal root ganglions (DRGs) on Gelatin/fNaCS. These figures illustrate confocal fluorescent images of DRGs with neurofilament stain (green) on gelatin, pNaCe1S or pNaCS, and fNaCe1S or fNaCS, respectively. The magnification is 4×, with a scale bar=250 μm.

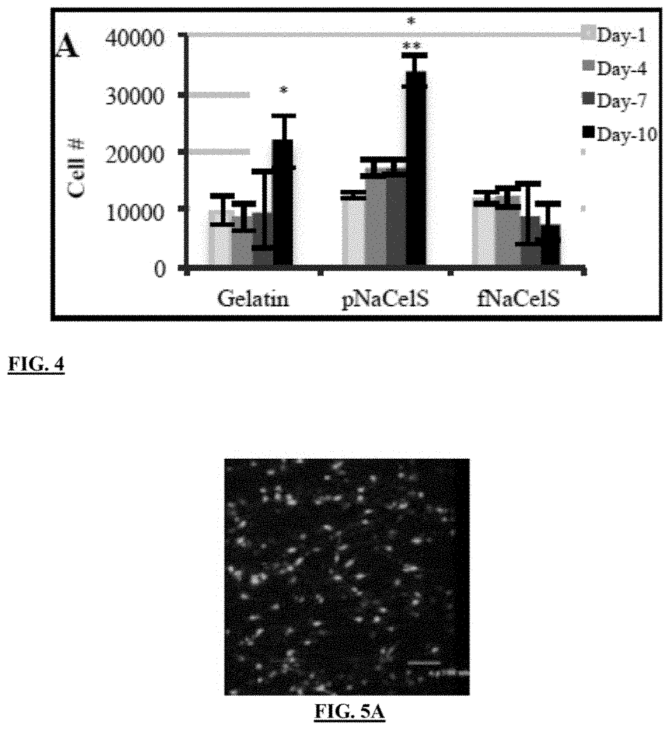

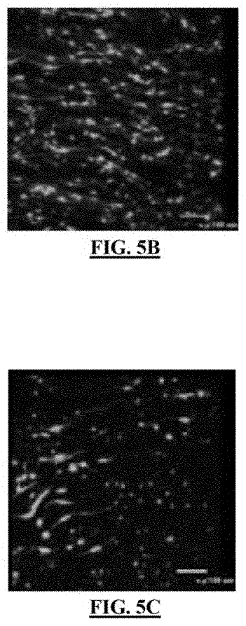

With reference to FIG. 4 and FIGS. 5A, 5B, and 5C, studies showed SCs attached at day 1 on all scaffolds. As shown, the SCs growth is up to 10 days. The sample size is n=4/time/group with a statistical significance of p<0.05 compared to day 1 and p<0.05 compared to all other groups and time points. Data shown are mean±Standard deviation. The confocal fluorescent images are of SCs at day 10 on Gelatin, pNaCS, and fNaCS, respectively at a magnification of 20×, and a scale bar=100 μm.

FIG. 6 shows rolling a scaffold longitudinally around a needle to form a spiral shape “conduit.” The schematic of FIG. 6 illustrates one embodiment of the fabrication of the Ce1S-Gelatin Conduits for in vivo studies. Aligned fibrous scaffolds are cut at the desired width depending on the desired final diameter of conduit. For this example, but not in a limiting manner, 1.5 mm width for the rat spinal cord and the desired length was made depending on the specific injured area or a clinician's determination. Again, depending on the embodiment, a 20G stainless steel needle (OD: 0.9 mm) was used to roll the scaffolds gently longitudinally around the needle to form a spiral shape conduit. The last layer is sealed longitudinally using DI-water to avoid unfolding during implantation. The needle was removed gently to avoid disturbing the fiber alignment. The conduit was crosslinked using 1-Ethyl-3-(3-dimethylaminopropyl) carbodiimide (EDC) in combination with N-hydroxysuccinimide (NHS), or depending on the embodiment sulfoNHS, crosslinking method employed for cros slinking for 96 hrs.

FIG. 7 is confocal fluorescent images of Ce1S-Gelatin or NaCS-Gelatin based conduits without Schwann cells having a sample size n of 6. Again, the abbreviation Ce1S means NaCS. The images are taken after 1 week of implantation in the rat transected spinal cord. Rostral interface at the top and caudal interface is at the bottom. Shown in the images are a GFAP (astrocyte marker) in green and a Neurofilament (axon marker) in red. Axons in the conduit appear to traverse the conduit as early as 1 week post-implantation.

Conclusions

As is evident from the results, a combination of SCs and GAG-mimetic scaffold can be used as a novel approach to effectively promote and direct axonal growth. The significant neurite growth on Gel/fNaCS suggests that neurite outgrowth can be promoted by the GAG mimetic and it may depend on the degree and pattern of sulfation. Again the results demonstrated in FIG. 7 in the complete transection model indicated axon regeneration as early as 1 week of implantation.

Furthermore, it was determined in these studies, that the conduit or rolled scaffold may be used alone or combination with Schwann cells (SCs) for spinal cord repair, and the like. In addition, the conduit also can be used for peripheral nerve repair, as well as in the methods and techniques described herein.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

What is claimed is:

1. A composition for a glycosaminoglycans (GAGs) mimetic scaffold, comprising:
a sodium cellulose sulfate (NaCS) scaffold containing aligned fibers; and wherein, the scaffold promotes spinal cord repair or peripheral nerve repair.

2. The composition of claim 1, further includes:
at least one Schwann Cell (SC) disposed about or in the scaffold; and wherein, the SCs stimulate axon growth.

3. The composition of claim 1, wherein the NaCS is either partial or fully sulfated and the scaffold mimics an in vivo spinal cord extracellular matrix (ECM).

4. The composition of claim 1, wherein the NaCS has a varying degree and a pattern of sulfation similar to a native GAGs for forming a GAGs mimetic scaffold.

5. The composition of claim 4, wherein the native GAGs is selected from a group consisting of chondroitin-6-sulfate or chondroitin sulfate-C (CS-C), chondroitin-2,6-sulfate or chondroitin sulfate-D (CS-D), and any combination thereof.

6. The composition of claim 4, wherein the GAGs mimetic scaffold is selected from a group consisting of a partial sulfated sodium cellulose sulfate (pNaCS), a fully sulfated sodium cellulose sulfate (fNaCS), and any combination thereof.

7. The composition of claim 6, wherein the partial sulfated sodium cellulose sulfate (pNaCS) contains a sulfate group on a 6th position of a glucose unit.

8. The composition of claim 6, wherein the fully sulfated sodium cellulose sulfate (fNaCS) contains a sulfate group on a 2nd, 3rd, and 6th positions of a glucose unit.

9. The composition of claim 8, further including at least one Schwann Cell (SC) disposed about or in the GAG mimetic scaffold for promoting and directing axonal growth.

10. The composition of claim 9, wherein the scaffold is fabricated using an electrospinning technique.

11. The composition of claim 1, wherein neurite growth on the scaffold depends on the degree and pattern of sulfation.

12. The composition of claim 1, wherein the average scaffold thickness is about 0.2mm

13. The composition of claim 1, wherein the scaffold is either a partial sulfated sodium cellulose sulfate (pNaCS) with 0.5 sulfates per glucose unit or a fully sulfated sodium cellulose sulfate (fNaCS) with 3 sulfates per glucose unit.

14. The composition of claim 13, wherein the scaffold further includes either gelatin or gelatin/polycaprolactone (Gel/PCL) at the ratio of 80:20 with a 0.25% concentration of fNaCS or pNaCS.

15. The composition of claim 1, wherein the scaffold further includes a crosslinked conduit to improve hydrolytic stability in a physiological condition.

16. A method for preparing a glycosaminoglycans (GAGs) mimetic scaffold, comprising:
dissolving a sodium cellulose sulfate (NaCS) conduit with a varying degree of sulfation in 15% deionized (DI) water to form a dissolved solution;
mixing the dissolved solution with a solution of either 100% bovine gelatin or a 80:20 ratio of gelatin: a poly-caprolactone (PCL) in an acetic acid (AA) and a 2,2,2-trifluoroethanol (TFE) to form a resultant solution;
electrospinning the resultant solution for preparing a scaffold containing aligned fibers; and
wherein, the scaffold promotes spinal cord repair or peripheral nerve repair.

17. The method of claim 16, further includes:
disposing at least one Schwann Cell (SC) about or in the scaffold; and
wherein, the SC stimulates extension of axons.

18. The method of claim 16, wherein the varying degree of sulfation includes a partial sulfated sodium cellulose sulfate (pNaCS) with 0.5 sulfates per glucose unit or a fully sulfated sodium cellulose sulfate (fNaCS) with 3 sulfates per glucose unit.

19. The method of claim 16, wherein the mixing of the 80:20 ratio of gelatin further includes:
preparing in the trifluoroethanol (TFE), the acetic acid (AA), and the deionized water at a ratio of 60:25:15 either a 25% w/v gelatin or a 22% w/v gelatin/polycaprolactone (Gel/PCL) at the ratio of 80:20 with a 0.25% of a fully sulfated sodium cellulose sulfate (fNaCS) or a partial sulfated sodium cellulose sulfate (pNaCS).

20. A method for preparing a glycosaminoglycans (GAGs) mimetic scaffold, comprising:
preparing a sodium cellulose sulfate (NaCS) scaffold containing aligned fibers;
disposing at least one Schwann Cell (SC) about or in the scaffold; the SC promotes axon growth;
wherein, the scaffold promotes spinal cord repair or peripheral nerve repair; and
wherein, the NaCS is either a partially sulfated sodium cellulose sulfate (pNaCS) with 0.5 sulfates per glucose unit or a fully sulfated sodium cellulose sulfate (fNaCS) with 3 sulfates per glucose unit at a concentration of fNaCS or pNaCS of 0.25%.
